(19) United States
(12) Patent Application Publication
(10) Pub. No.: US 2003/0060872 A1
(43) Pub. Date: Mar. 27, 2003

(54) Stent with radiopaque characteristics

(76) Inventors: Gary Gomringer, Letterkenny (IE); Thomas Trozera, Del Mar, CA (US)

Correspondence Address: FISH & RICHARDSON PC, 225 FRANKLIN ST, BOSTON, MA, 02110, US

(21) Appl. No.: 09/963,817

(22) Filed: Sep. 26, 2001

(51) Int. Cl. A61F 2/06

(52) U.S. Cl. 623/1.15

(57) ABSTRACT

This invention provides for radiopaque rivets to be attached to radially expandable surgical stents. Such rivets are attached at various points of the stent which will allow these points to be readily viewable by a fluoroscope or other imaging device. The rivets are formed of a material having radiopaque characteristics and attached to the stent in a manner that would allow the stent to function normally. Each rivet can be made using a variety of malleable, non-corrosive, and radiopaque metals such as gold, platinum, osmium, palladium, rhenium, tantalum, or tungsten. It is also contemplated that any combination of these radiopaque materials can be used to fabricate the rivet.

### BACKGROUND OF THE INVENTION

The following invention relates to radially expandable stents for implantation into a body lumen, such as an artery. More specifically, this invention relates to radially expandable surgical stents which are adapted to include radiopaque rivets thereon to enhance their visibility when viewed with an imaging device such as a fluoroscope.

Surgical stents have long been known which can be surgically implanted into a body lumen, such as an artery, to reinforce, support, repair, or otherwise enhance the performance of the lumen. For instance, in cardiovascular surgery, it is often desirable to place a stent in the coronary artery at a location where the artery is damaged or is susceptible to collapse. The stent, once in place, reinforces that portion of the artery allowing normal blood flow to occur through the artery. One form of stent which is particularly desirable for implantation in arteries and other body lumens is a cylindrical stent which can be radially expanded from a first smaller diameter to a second larger diameter. Such radially expandable stents can be inserted into the artery by being located on a catheter and fed internally through the arterial pathways of the patient until the unexpanded stent is located where desired. The catheter is fitted with a balloon or other expansion mechanism which exerts a radial pressure outward on the stent causing the stent to expand radially to a larger diameter. Such expanded stents exhibit sufficient rigidity after being expanded that they will remain expanded after the catheter has been removed.

Radially expandable stents come in a variety of different configurations to provide optimal performance in various different particular circumstances. For instance, the United States patents to Lau (U.S. Pat. Nos. 5,514,154, 5,421,955, and 5,242,399), Barracci (U.S. Pat. No. 5,531,741), Gaturud (U.S. Pat. No. 5,522,882), Gianturco (U.S. Pat. No. 5,507,771 and 5,314,444), Termin (U.S. Pat. No. 5,496,277), Lane (U.S. Pat. No. 5,494,029), Maeda (U.S. Pat. No. 5,507,767), Marin (U.S. Pat. No. 5,443,477), Khosravi (U.S. Pat. No. 5,441,515), Jessen (U.S. Pat. No. 5,425,739), Hickle (U.S. Pat. No. 5,139,480), Schatz (U.S. Pat. No. 5,195,984), Fordenbacher (U.S. Pat. No. 5,549,662), and Wiktor (U.S. Pat. No. 5,133,732), each include some form of radially expandable stent for implantation into a body lumen.

A major difficulty which has surfaced in the use of such surgical stents has been the difficulty in determining the precise location of the stent both during and after implantation. This is due to the fact that materials commonly used in the production of surgical stents such as stainless steel or nickel titanium alloys are not readily perceptible when the treated site is viewed with fluoroscopes or other such medical imaging devices.

The prior art teaches several methods that have been developed to provide for varying amounts of radiopacity of surgical stents. For instance, radiopaque markers have been added to stents to provide a clearly identifiable point of reference easily viewed through fluoroscopy or other medical imaging technology. Unfortunately, such visibility has come at the cost of the effectiveness of the stent as the addition of these markers has adversely affected the ability of the stent to expand properly. There has also been difficulty with such markers protruding from the surface of the stent. This could cause damage to the arterial wall or impede blood flow through the stent and add to the likelihood of restenosis. Another method has been to apply a radiopaque coating to portions of a surgical stent. The increased thickness of the stent at the points where it was coated again interfered with the proper function of the stent. Moreover, the possibility exists that the plating material could become detached from the stent and prove a threat to the safety of the patient.

### SUMMARY OF THE INVENTION

This invention provides for radiopaque rivets to be attached to radially expandable surgical stents. Such rivets are attached at various points of the stent which will allow these points to be readily viewable by a fluoroscope or other imaging device. Rather than utilizing such methods as overlaying non-radiopaque stent elements with a coating of radiopaque material or attaching a radiopaque element to a stent in a manner that would interfere with its function, the rivets are formed of a material having radiopaque characteristics and attached to the stent in a manner that would allow the stent to function normally.

Each rivet can be made using a variety of malleable, non-corrosive, and radiopaque metals such as gold, platinum, osmium, palladium, platinum, rhenium, tantalum, or tungsten. It is also contemplated that any combination of these radiopaque materials can be used to fabricate the rivet.

The rivet is fabricated from wire or similar structure with an appropriate diameter that is melted or machined at one end in such a fashion as to create a bulbous, beveled design, “T” configuration, or other appropriately shaped distal head which is part of the original metal. The wire or similar structure is cut to size for the particular application. Then this rivet is placed through an appropriately configured hole in a stent strut, passing from the interior to the exterior of the stent. The distal head of the rivet prevents the unit from completely sliding through the hole. The distal head is fitted into the hole preferably from the inner surface of the stent, thereby creating a smooth interior surface within the stent. Alternately, the distal head can be fitted into the hole from the outer surface of the stent. The rivet is engaged or locked into place through compression or upsetting of the protruding proximal portion of the rivet causing it to take the form of a flattened head on the exterior surface (or alternatively, the interior surface) of the stent. Optionally, a washer mechanism can be inserted over the shaft of the rivet on either the distal head, proximal portion, or both, prior to compression, to provide a larger base for securing the rivet to the stent.

Accordingly, a primary object of the present invention is to provide a radially expandable surgical stent which features radiopaque rivets attached thereto which enhance the visibility of the stent when viewed through a fluoroscope or other imaging device.

Another object of the present invention is to provide a radially expandable surgical stent with radiopaque rivets that do not inhibit radial expansion and support of a body lumen by having the radiopaque rivets included thereon.

Another object of the present invention is to provide radiopaque rivets for a surgical stent which attach to various points on the stent and which are formed from a radiopaque material.

Another object of the present invention is to provide a stent with radiopaque rivets which are distinctly visible when viewed with an imaging device, such as a fluoroscope, but do not obscure other structures located adjacent to the radiopaque rivets.

Another object of the present invention is to develop a method for attaching radiopaque rivets to various points on the stent.

Other further objects of the present invention will become apparent from a careful reading of the detailed description of the preferred embodiments, the claims, and the drawing figures included herein.

### BRIEF DESCRIPTION OF THE DRAWINGS

FIG. 1 is a schematic view of the present invention in its intended operational environment.

FIG. 2 is an isometric rendering of a prior art surgical stent such as would be used in conjunction with radiopaque rivets.

FIG. 3 is a top view of an optional rivet washer that is used to mount the rivet to the stent.

FIG. 4 is a side view of a typical radiopaque rivet.

FIG. 5 is a side view of the alternate washer, taken on section lines 5-5 of FIG. 3 that is designed to engage the distal head of the radiopaque rivet.

FIG. 6 is an isometric rendering of the radiopaque rivet as it appears prior to installation in the surgical stent demonstrating the optional washer designed to be engaged to the proximal end of the radiopaque rivet.

FIG. 7a is a fragmentary isometric rendering of a portion of a surgical stent showing the holes in the stent struts designed to receive the radiopaque rivets positioned on the inside of the stent.

FIG. 7b is a fragmentary isometric rendering of a portion of a surgical stent showing initial insertion of radiopaque rivets.

FIG. 7c is a fragmentary isometric rendering of a portion of a surgical stent showing the form and position of radiopaque rivets after installation is complete.

FIG. 8 is a fragmentary isometric figure demonstrating a more detailed example of the hole and one configuration of the radiopaque rivet.

FIG. 9 is a fragmentary isometric figure demonstrating a more detailed example of the radiopaque rivet position within the hole in the stent strut.

FIG. 10 is a fragmentary isometric figure demonstrating a more detailed example of the hole and another configuration of the radiopaque rivet.

FIG. 11 is a fragmentary isometric figure demonstrating a more detailed example of the alternate radiopaque rivet position within the hole in the stent strut with the proximal end compressed forming a secondary rivet head.

FIG. 12A is a schematic view of the present invention in its intended operational environment demonstrating the stent with radiopaque characteristics proximal to the lesion with the representative fluoroscope (cine) not showing the stent with the lesion.

FIG. 12B is a schematic view of the present invention in its intended operational environment demonstrating the stent with radiopaque characteristics in a contracted configuration, centered within the lesion and a representative fluoroscope (cine) showing the relative location of the rivets, and therefore the stent, within the lesion.

FIG. 12C is a schematic view of the present invention in its intended operational environment demonstrating the stent with radiopaque characteristics in an expanded configuration, centered within the lesion and a representative fluoroscope (cine) showing the relative location of the rivets, and therefore the stent, within the lesion.

FIG. 12D is a schematic view of the present invention in its intended operational environment demonstrating the stent with radiopaque characteristics, deployed within the lesion with delivery balloon retracted, and the representative fluoroscope (cine) showing location of the rivets, and therefore the stent, within the lesion.

### DESCRIPTION OF THE PREFERRED EMBODIMENT

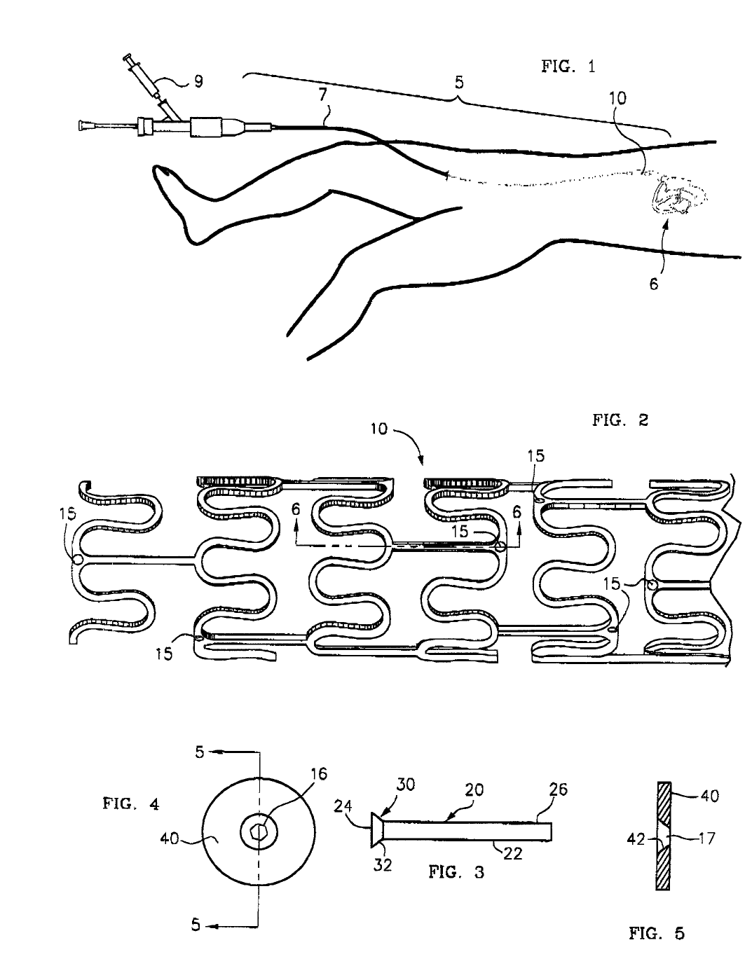

As illustrated in FIGS. 1 and 2, the surgical stent with radiopaque rivets system 5 embodying features of the invention is comprised of a surgical stent 10 which has been adapted to accept installation of radiopaque rivets 20 through holes 15 in various struts 12 of the stent. A delivery catheter 7 with guide wire support and inflation lumens is also considered as part of the system 8. Also shown is an inflation-deflation device 9 used to inflate an expandable member (balloon) on the distal end of the catheter to expand the present invention stent and deploy it within a lesion. The radiopaque rivets 20 would enable determination of the position of the stent within a patient's vascular system through the use of a fluoroscope or other imaging device.

As shown in FIG. 2, the construction of the system 5 begins with a surgical stent 10 as taught in the prior art. It is anticipated that this invention could be applied to surgical stents of varying configurations or designs. Depending upon which portions of the stent one desires to locate through fluoroscopy or other imaging technology, holes 15 would be placed in various struts or expandable members 12 of the stent 10. A couple of holes are shown in FIG. 2, however, it is contemplated by the present invention to have one or more, or a plurality of holes for radiopaque rivet insertion. Furthermore, some stent designs might have portions more adaptable for placing the holes 15 and radiopaque rivets 20 within the struts, backbone or expandable members in the stent 10. These holes 15 could be created during or after the production of the stent 10 by a number of means, for example, standard drilling technology, laser and enhance laser cutting techniques, or wire electrical discharge machining (EDM). It should be understood by one skilled in the art that other methods may be employed to create the holes in the stent struts. In order to provide a suitable anchor for a rivet head 24, these holes 15 would have an interior diameter slightly larger than the exterior diameter of the rivet shaft 22 (see FIG. 3).

Rivet 20 could be comprised of a variety of soft, malleable, non-corrosive, and radiopaque materials such as gold, osmium, palladium, platinum, rhenium, tantalum, and tungsten would consist of a length of wire of a suitable diameter comprised of such material and creating at one end a beveled or ball shaped head 24 and at the other a stem or shaft 22 such that the whole is a sample of the radiopaque material. The head 24 of the rivet 20 can be created by a number of means, for example, melting a length of wire in such a fashion as to create a ball shape which is part of the original metal, or machining the head 24 of the rivet 20 so that a beveled head design is produced. It should be understood by one skilled in the art that other methods may be employed to create the rivet and its associated sub-parts. In use with a stent having struts with a width in the range of 0.004 to 0.006 inches and a thickness in the range of 0.003 to 0.006 inches, for example, the rivet might have a head 24 with a diameter in the range of 0.0038 to 0.0058 inches and a stem 22 with a length in the range of 0.004 to 0.007 inches. In this case, the hole 15 might have an interior diameter 16 in the range of 0.0035 to 0.0055.

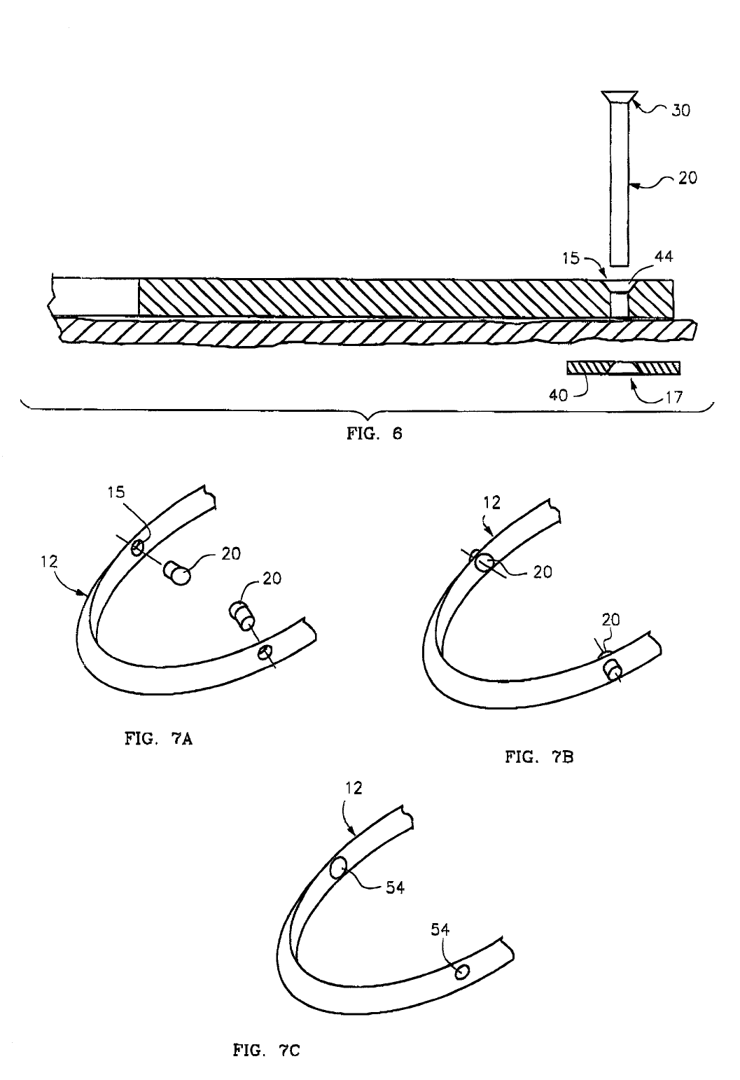

Now referring to FIGS. 3-6, radiopaque rivets 20 could be placed in holes 15 throughout stent 10 or an optional washer 40 in such a fashion that the exterior lip of said holes would allow the rivet stem 22 to pass through the stent strut 12 or washer 40, but would prevent rivet head 24 from doing so. The material comprising rivet head 24 could then be compressed into hole 15 in such a fashion that beveled rivet head 30 would completely fill hole 15 such that exterior surface 32 of such beveled rivet head 30 would be flush with the inside surface of the stent strut 12.

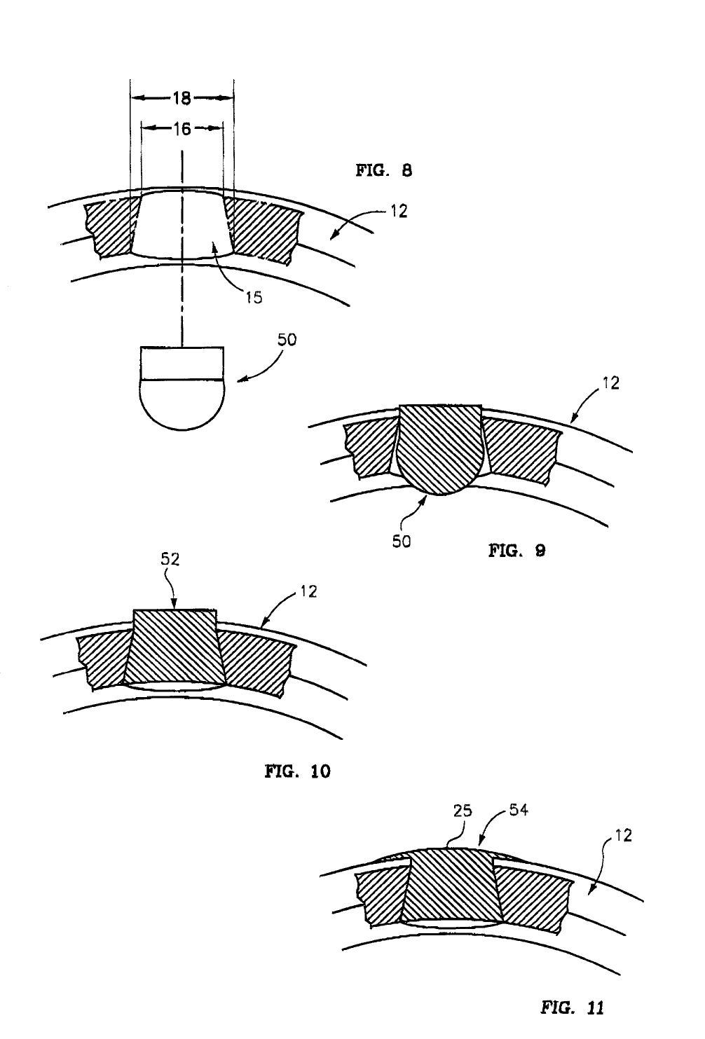

FIGS. 7a through 7c demonstrate the process of inserting a radiopaque rivet 20 into the hole 15 of a strut 12. In FIG. 7a, the rivet 20 is positioned such that distal head 24 is on the inside of the stent where the blood flow channel is located. Alternatively, the rivet 20 can be positioned such that the distal head 24 is on the exterior side of the stent. In FIG. 7b, the head is engaged and butted up against the interior surface of the hole 15 in the strut 44 such that a tight fit is obtained when compression is applied to the proximal end 26 of the shaft 22. FIG. 7c demonstrates the stage with the proximal end 26 of rivet stem 22 is compressed or upset in such a fashion that it would form secondary rivet head 25. The diameter 27 of such secondary rivet head 25 would be larger than the exterior diameter 18 of hole 15, thereby securing or locking rivet 20 in place (see FIGS. 7c & 10).

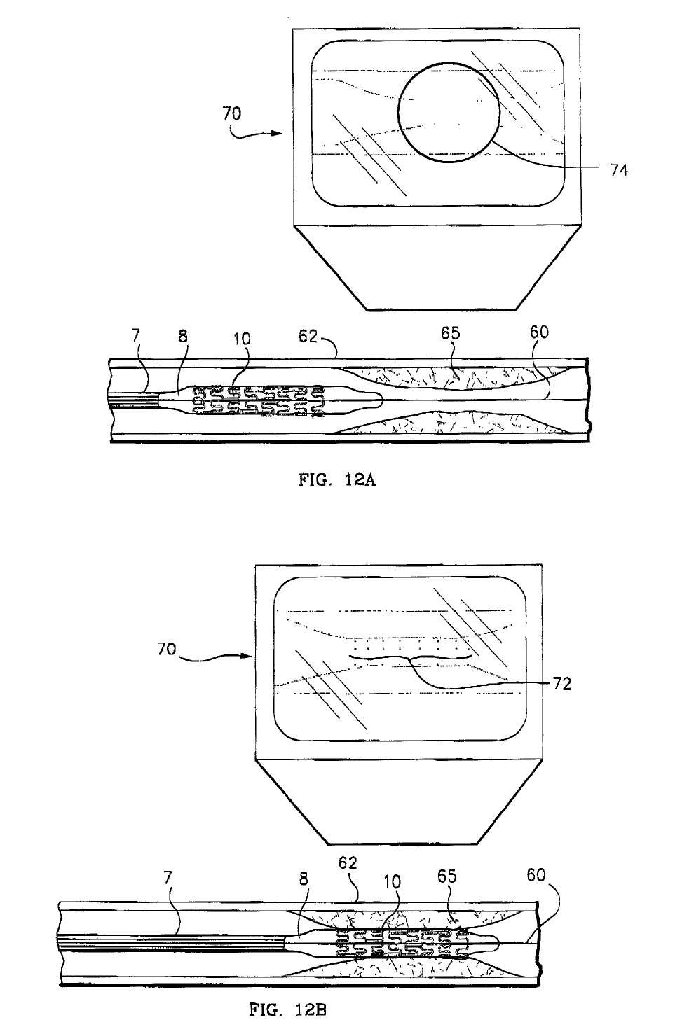

Now referring to FIGS. 8-11, various designs or configurations could be employed to function as the radiopaque rivet of the present invention. FIG. 8 and 9 show a bulbous head design 50 whereas FIG. 10 demonstrates a tapered or beveled design 52. It is contemplated that various other rivet head configurations could be employed to provide the fitting and securing characteristics shown by the previous figures.

FIG. 11 shows the tapered or beveled rivet head embodiment 52 after the manipulation or compression process has been complete to form a secondary head 25. The secondary head has been compressed such that the diameter of the head is larger than the hole 15, thereby functioning in association with the distal head, to firmly secure the rivet 20 within the hole 15 of stent 10.

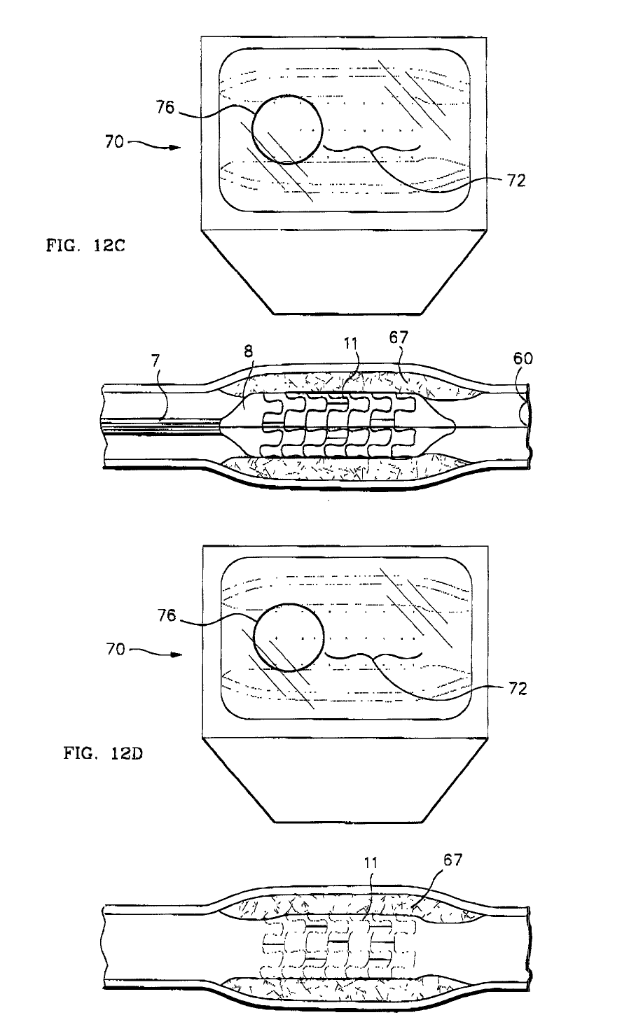

Now referring to the series of drawings presented in FIGS. 12a through 12d, the present invention system 5 comprising the stent 10 with radiopaque rivets 20 is mounted on the expandable member located on the distal end of a delivery catheter. In FIG. 12a, a guidewire 60 is inserted such that its distal tip is positioned distal to the lesion 65 in blood vessel 62. In the standard practice, a radiopaque dye is injected into the patient's vasculature just prior to observation on the fluoroscope or cine 70. In FIG. 12a, the contour of the vessel with the two dimensional estimation of the lesion morphology 74 is presented on the fluoroscope. Since the present invention system 5 is proximal to the lesion, its radiopaque rivets are not observed.

In FIG. 12b, the present invention system 5 has been advanced so that the stent with radiopaque rivets 10 and delivery balloon 8 are centered within the lesion 65 to be treated. The guidewire is still in a proximal position and the stent is in its contracted configuration. A representation of the radiopaque rivets 72 is shown in the fluoroscope 70. When radiopaque dye is injected into the patient's vasculature, the outline of the lesion and the relative position of the stent can be visualized. This visualization provides the clinician with beneficial clinical information, verifying that the stent is centered within the lesion to be treated prior to expansion and embedment into the vessel.

In FIG. 12c, the present invention system 5 remains such that the stent with radiopaque rivets 10 and delivery balloon 8 are centered within the treated lesion 67. The guidewire is still in a proximal position and the stent has been expanded and embedded into the vessel wall. A representation of the radiopaque rivets 72 is shown in the fluoroscope 70. The representation is different from that of FIG. 12b because in expanding the stent, the engaged radiopaque rivets have also moved towards, and in some cases, into the vessel wall. When radiopaque dye is injected into the patient's vasculature, the outline of the lesion and the relative position of the stent can be visualized. This visualization provides the clinician with beneficial clinical information, verifying that the stent was centered within the lesion treated and whether additional interventional treatment is necessary.

In FIG. 12d, the delivery catheter and expandable balloon have been retracted proximally. The stent with radiopaque rivets is deployed and at least partially embedded into the vessel wall. A representation of the radiopaque rivets 72 is shown in the fluoroscope 70. The representation is similar to that of FIG. 12c wherein the rivets appear to be relatively close to or embedded within the vessel wall. When radiopaque dye is injected into the patient's vasculature, the outline of the lesion and the relative position of the stent again can be visualized. This visualization provides the clinician with beneficial clinical information. During or subsequent to the primary interventional procedure, the clinician will always have evidence of the relative position of the stent within the vessel.

We claim:

1. A stent with radiopaque characteristics comprising:
said stent as taught in the prior art, said stent having a plurality of expandable members;
one or more holes in one or more of the expandable members of said stent;
one or more radiopaque rivets, said rivets mounted within said holes.

2. A stent with radiopaque characteristics as recited in claim 1, wherein said rivet is fabricated from gold.

3. A stent with radiopaque characteristics as recited in claim 1, wherein said rivet is fabricated from osmium.

4. A stent with radiopaque characteristics as recited in claim 1, wherein said rivet is fabricated from palladium.

5. A stent with radiopaque characteristics as recited in claim 1, wherein said rivet is fabricated from platinum.

6. A stent with radiopaque characteristics as recited in claim 1, wherein said rivet is fabricated from rhenium.

7. A stent with radiopaque characteristics as recited in claim 1, wherein said rivet is fabricated from tantalum.

8. A stent with radiopaque characteristics as recited in claim 1, wherein said rivet is fabricated from tungsten.

9. A stent with radiopaque characteristics as recited in claim 1, wherein said rivet is fabricated from a group consisting of gold, osmium, palladium, platinum, rhenium, tantalum, or tungsten.

10. A stent with radiopaque characteristics as recited in claim 1, wherein said rivet is fabricated from any combination of the group consisting of gold, osmium, palladium, platinum, rhenium, tantalum, or tungsten.

11. A stent with radiopaque characteristics comprising:
a substantially cylindrical framework, said framework having a plurality of struts;
one or more holes in one or more struts of said framework;
one or more radiopaque rivets, said rivets mounted within said holes;

12. A stent with radiopaque characteristics as recited in claim 11, wherein said rivet is fabricated from gold.

13. A stent with radiopaque characteristics as recited in claim 11, wherein said rivet is fabricated from osmium.

14. A stent with radiopaque characteristics as recited in claim 11, wherein said rivet is fabricated from palladium.

15. A stent with radiopaque characteristics as recited in claim 11, wherein said rivet is fabricated from platinum.

16. A stent with radiopaque characteristics as recited in claim 11, wherein said rivet is fabricated from rhenium.

17. A stent with radiopaque characteristics as recited in claim 11, wherein said rivet is fabricated from tantalum.

18. A stent with radiopaque characteristics as recited in claim 11, wherein said rivet is fabricated from tungsten.

19. A stent with radiopaque characteristics as recited in claim 11, wherein said rivet is fabricated from a group consisting of gold, osmium, palladium, platinum, rhenium, tantalum, or tungsten.

20. A stent with radiopaque characteristics as recited in claim 11, wherein said rivet is fabricated from any combination of the group consisting of gold, osmium, palladium, platinum, rhenium, tantalum, or tungsten.

21. The method of fabricating a stent with radiopaque characteristics;
creating a plurality of holes in one or more struts of such stent;
mounting radiopaque rivets in said holes.
